(19) United States
(12) Patent Application Publication
(10) Pub. No.: US 2007/0197959 A1
(43) Pub. Date: Aug. 23, 2007

(54) Endotherapy Catheter

(75) Inventor: Christos Panotopoulos, Athens (GR)

Correspondence Address: THE WEBB LAW FIRM, P.C., 700 KOPPERS BUILDING, 436 SEVENTH AVENUE, PITTSBURGH, PA, 15219, US

(73) Assignee: Microdialysis EPE, Athens (GR)

(21) Appl. No.: 10/570,403

(22) PCT Filed: Sep. 3, 2004
(86) PCT No.: PCT/GR04/00045

§ 371 Date: Oct. 6, 2006

(30) Foreign Application Priority Data
Sep. 8, 2003 (GR) 20030100371

(51) Int. Cl. A61M 31/00; A61M 37/00

(52) U.S. Cl. 604/93.01

(57) ABSTRACT

A catheter that can be used for concurrent fluid infusion and aspiration in humans, animals and biological material, at a wide range of flow rates, without any blockage problems. The catheter is composed of two concentric tubes; their proximal ends are properly connected to the infusion equipment and the aspiration equipment respectively; the distal end of the catheter is covered by a filter or membrane or grid or mesh cage and contains a hydrodynamically moving device of concurrent infusion and aspiration. The inner tube is properly assembled to the moving infusion and aspiration device, which irrigates the space surrounding the catheter's tip, through the filter or membrane or grid or mesh cage cover preserving its permeability, while it helps, due to its motion, the aspiration through the outer tube.

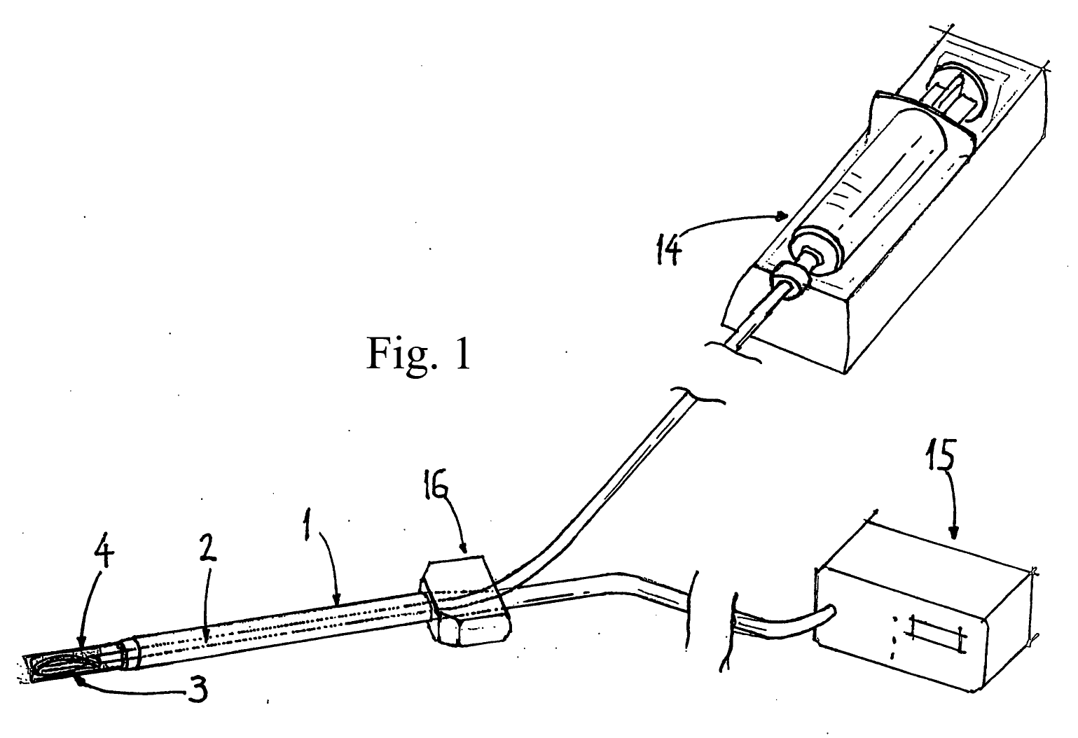

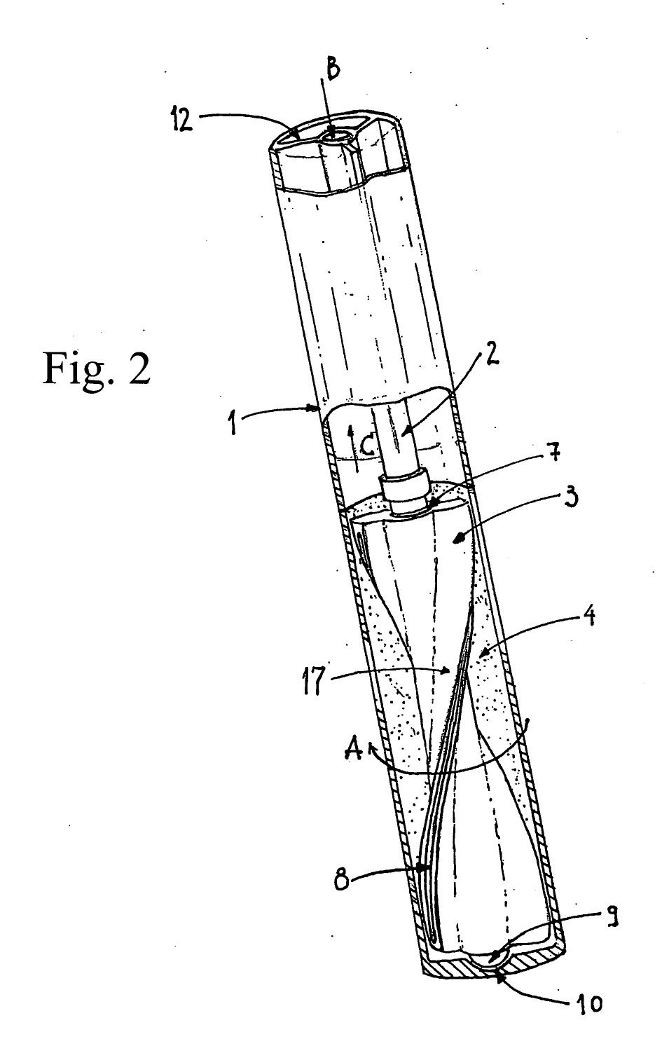

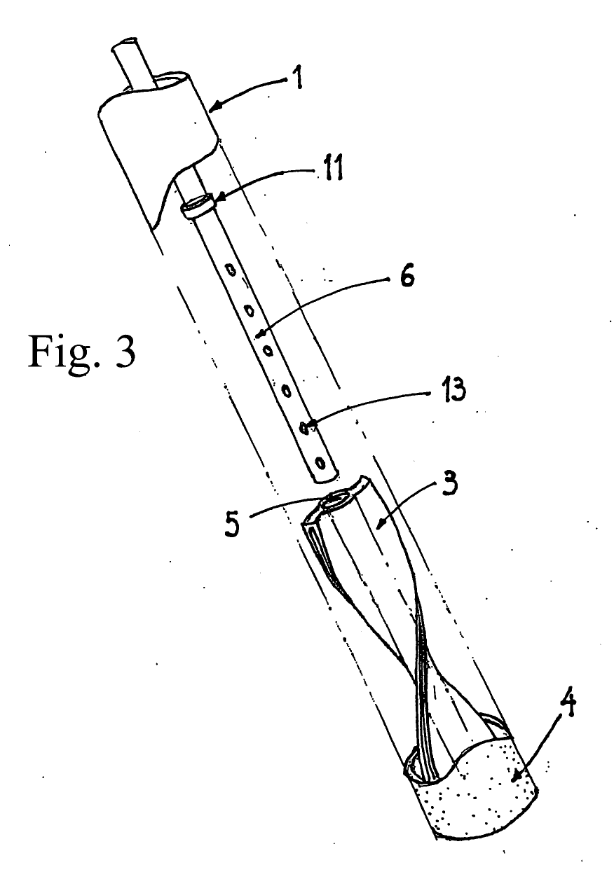

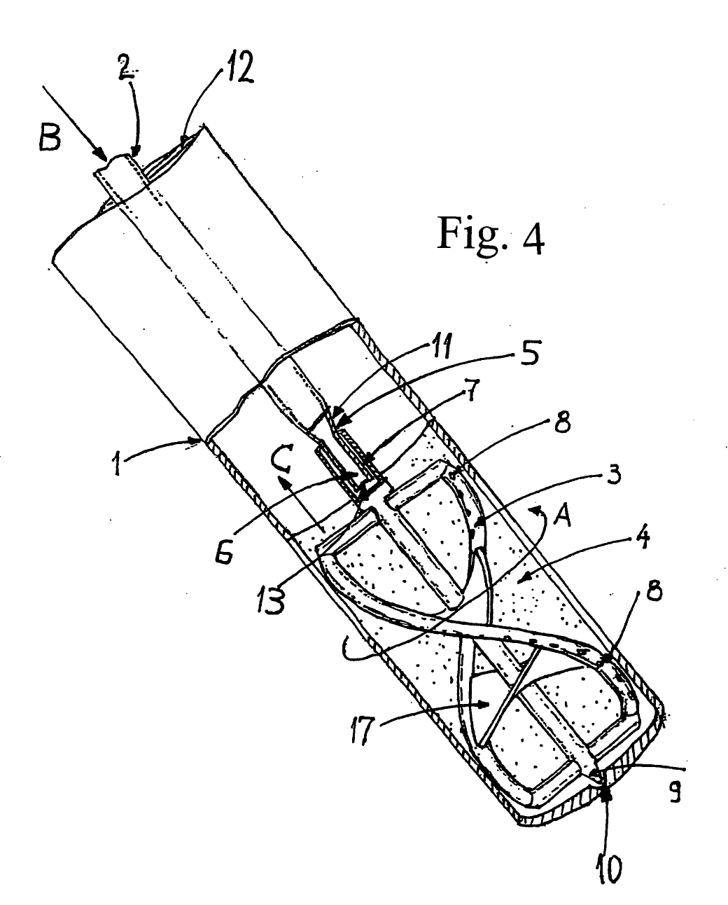

The proposed invention is a catheter that can be used for infusion of drugs and nutrients with concurrent aspiration of biological material, in human and, or, animal tissue and, or, body cavity, and, or, neoplastic tissue and, or, pathological liquid accumulations in the body.

There are many kinds of catheters which are used for fluid infusion and aspiration in a clinical or preclinical setting.

Traditionally, the catheter's tip that is inserted in biological material, is called “distal” and the tip that stays outside is called “proximal”.

Most of existing catheters have a single lumen-tube and through this lumen-tube the user—doctor, nurse, scientist or laboratory personnel—can alternatively infuse or aspirate liquids.

For example, in a clinical setting, the common intravenous catheter either aspirates blood samples—usually immediately after it's insertion to the vein—or infuses solutions of drugs and, or, nutrients—usually for many hours or days following insertion.

These catheters can infuse or aspirate large quantities of liquids, but they cannot do it concurrently in order to have a constant exchange of drugs and nutrients with pathological liquid accumulations.

That means that during the infusion phase, the tissue increases in volume and this could be dangerous or even fatal in certain cases (for example in an already suffering from oedema brain).

There are also catheters with multiple lumen-tubes, which can concurrently infuse and aspirate liquids.

For example, the microdialysis catheter after it's introduction to a human or animal tissue, is continuously perfused with liquid solutions from a pump connected to its proximal tip. The catheter consists of two concentric lumens-tubes, that are covered at their distal tip by a membrane. Usually the central lumen-tube is the efferent and the peripheral lumen-tube is the afferent part of the catheter. Part of the perfused liquid is infused to the tissue through the catheter's membrane at its distal end, and extracellular tissue fluid is aspirated through the same membrane and the efferent lumen-tube.

Microdialysis catheters and similar to them catheters though, were designed for tissue monitoring, and the above described concurrent infusion and aspiration takes place at a microliters flow rate. For therapeutic applications we need greater liquid exchange rate.

Additionally, a common problem of all kinds of existing catheters for biological fluids, is their blockage, due to corking of biological material into their lumen's aspirating tip, or coverage of their liquid exchange membrane (like microdialysis catheter's membrane) from organic substances (mostly proteins).

The proposed endotherapy catheter infuses and aspirates, even great quantities of liquids, concurrently, at a wide range of flow rates, without any blockage problems.

It consists of two concentrical lumens-tubes, connected properly to infusion and aspiration devices at their proximal tip, and having a filter or membrane or grid or mesh cage covering their distal tip, which contains an hydrodynamically moving device for concurrent infusion and aspiration.

The infusing lumen-tube is appropriately connected to the device that irrigates the surrounding the catheter space, while simultaneously propels with its movement the aspiration through the other tube.

The endotherapy catheter utilizes the circulating fluid's shear forces to remove any biological material that blocks the catheter's distal tip.

The attached drawings represent two of the many possible variations of the endotherapy catheter.

The numbers and letters of the drawings refer to:
1) aspiration outer lumen-tube
2) infusion inner lumen-tube
3) moving-rotating device
4) liquid exchange surface
5) moving-rotating device's port-housing for stator
6) stator
7) intermediate space between stator and moving-rotating device
8) moving-rotating device's ports-openings
9) moving-rotating device's tip
10) housing for the moving-rotating device's tip
11) inner lumen-tube's travel limiter
12) centering supports
13) stator's through holes-openings
14) infusion device
15) aspiration device and, or, collection tank and, or, analysis device
16) catheter bifurcation
17) proximal face of the moving-rotating device
A) Direction of movement-rotation of the moving-rotating device
B) Direction of infused liquid
C) Direction of aspirated liquid

The endotherapy catheter has an infusion inner lumen-tube (2) and an aspiration outer lumen-tube (1). The fluid is supplied by an infusion device (14) or any liquid container that has positive pressure, relatively to the pressure of the surrounding the catheter's tip tissue, while the returning fluid is collected by a negative pressure pump, or any liquid container with negative pressure, relatively to the pressure of the surrounding the catheter's tip tissue.

The endotherapy catheter has a bifurcation part (16), in order to split the two opposite flows in two different lumens-tubes, as shown in drawing 1.

The distal end of the outer lumen-tube holds an exchange surface (4), that can be a filter or membrane or grid or mesh cage.

Fluid, which can vary from distilled water to nutrient solutions with drugs, that is supplied through the inner lumen-tube (2), according to arrow B, reaches the distal end of the catheter, where substance exchange occurs between the infused fluid and substances contained in the surrounding tissue's extracellular fluid; the fluid returns to an aspiration device and, or, collection tank and, or, measurement system (15), according to arrow C.

In order to remove organic substances that are built up on the exchange surface, and consequently block the catheter, a fluid jet, receiving its supply from the inner lumen-tube (2), is dispersed against the liquid exchange surface's inner wall (4), via the moving-rotating device's ports (8), as shown in drawings 2, 4. The jet propels the rotation of the moving-rotating device (3) according to arrow A.

Drawings 2, 3 and 4 depict two of the many possible variations of the same concept. In the first variation, shown in drawings 2, 3, the moving-rotating device has a hollow twisted plate shape, while in the second variation, shown in drawing 4, the moving-rotating device resembles a twin helix chain.

As shown in drawing 3, the moving-rotating device (3) holds a port (5) that serves as a fluid supply inlet, but also as a housing for the stator (6), which is the distal end of the inner lumen-tube (2).

The stator (6) may hold, circumferentially and on its end, through holes-openings (13), to allow fluid outlet from the inner lumen-tube (2) to the intermediate space (7) between stator and moving-rotating device. This intermediate space is created since the stator's (6) outer diameter is slightly smaller than the moving-rotating device's port (5) diameter, and serves as a mass transfer subspace and a friction eliminator, since it follows a slide bearing function principal.

The moving-rotating device (3) may have an helical shape and hold ports-openings (8), that take fluid from the intermediate space between stator and moving-rotating device (7), and redirect it against the exchange surface walls (4), with a direction angle other than the radial, so that a rotational propulsion is achieved, as shown in drawings 2, 4.

The angle is selected based on a trade-off between the device's (3) rotation frequency and the shear stress on the exchange surface walls.

That is, a rather radial direction biased angle selection would result on fewer rotations per given time but higher shear stresses, while a rather circumferential direction biased angle selection would result on more rotations per given time but lower shear stresses.

Therefore, the moving-rotating device (3) not only removes the organic remains that block the exchange surface (4), but is also responsible for its movement-rotation.

As shown in drawings 2, 4, the moving-rotating device (3) may have an overall or particular helical shape with a spin direction such that, due to the jet-induced rotation, its proximal face (17) pushes fluid proximally, forcing its return to the extracorporeal collecting equipment.

This is particularly useful to avoid stagnation of the organic substances that were exchanged through the filter or membrane or grid or mesh cage, by forcing their removal.

As shown in drawings 2, 4, the tip (9) of the moving-rotating device could be such that it supports the device in place inside the outer tube (1) and at the same time allows for relative movement-rotation. To facilitate that, the lower part of the outer tube may hold a recess (10), in order to house the tip (9) of the moving-rotating device (3).

In addition, a travel limiter (11) can be present at an appropriate level of the inner tube, to assure operation under all inclinations.

The inner tube (2) may be centered coaxially to the outer tube (1) to ensure evenness in function. To achieve that, one or more centering supports (12) can be placed between the inner and outer tubes, just proximally to the moving-rotating device (3) level.

The catheter may have an overall flexibility in order not to present resistance during any movement of the implanted tissue relatively to its, relatively stable, exit point, however the distal end has to be fairly rigid, to ensure that the moving-rotating part can work properly.

So, the materials are selected appropriately, to offer relative stiffness at the distal end of the inner and outer tube, while more compliant materials may be selected for the rest of the catheter.

For certain clinical and laboratory applications though, the whole catheter can be rigid.

The material of the catheter should also be in conformity to the norms and regulations existing for clinical and laboratory catheters, including biocompatibility issues etc.

1. An endotherapy catheter, comprising an infusion lumen-tube connected at a proximal end to a pump, bottle or any apparatus that contains fluids for infusion under positive pressures, an aspiration lumen-tube connected at a proximal end to a pump, bottle or any apparatus that collects fluids under negative pressures, of a filter, membrane, grid or mesh cage positioned at a distal end of the catheter, through which infusion and aspiration takes place, and of a hydrodynamically moving device, positioned inside the filter, membrane, grid or mesh cage, which directs infused fluid, through the surface of the filter, membrane, grid or mesh cage, to the catheter's surrounding space, thereby preventing biological material deposition and consequent blockage of a distal catheter tip, and simultaneously, due to its motion, improving the aspiration of the fluid surrounding the catheter.

2. The endotherapy catheter according to claim 1, wherein the hydrodynamically moving device is in a helical shape at its distal end.

3. The endotherapy catheter according to claim 1, wherein the hydrodynamically moving device exhibits rotational movement at its distal end.

4. The endotherapy catheter according to claim 1, wherein the infusion lumen is moveable.

5. The endotherapy catheter according to claim 4, wherein the infusion lumen is moveable electromethanically, mechanically, through placement of a moving device at its distal end, or any combination thereof.

6. The endotherapy catheter according to claim 2, wherein the hydrodynamically moving device exhibits rotational movement at its distal end.

7. The endotherapy catheter according to claim 2, wherein the infusion lumen is moveable.

8. The endotherapy catheter according to claim 3, wherein the infusion lumen is moveable.

9. The endotherapy catheter according to claim 7, wherein the infusion lumen is moveable electromethanically, mechanically, through placement of a moving device at its distal end, or any combination thereof.

10. The endotherapy catheter according to claim 8, wherein the infusion lumen is moveable electromethanically, mechanically, through placement of a moving device at its distal end, or any combination thereof.

11. The endotherapy catheter according to claim 1, wherein the catheter is used in a clinical setting, a pre-clinical setting, a laboratory, or any combination thereof.
